(19) United States
(12) Patent Application Publication
(10) Pub. No.: US 2012/0214212 A1
(43) Pub. Date: Aug. 23, 2012

(54) Expression and application of recombinant FAEES in algae as a novel method to produce biodiesel in vivo

(75) Inventors: Chifu Huang, Lexington, KY (US); Jeffrey Lee Ebersole, Lexington, KY (US)

(73) Assignee: Dr. Chifu Huang, Lexington, KY (US)

(21) Appl. No.: 13/029,597

(22) Filed: Feb. 17, 2011

(51) Int. Cl. C12P 7/64

(52) U.S. Cl. 435/134

(57) ABSTRACT

This discovery is related to the production of bioenergy fuels in vivo, specifically related to the production of biodiesel fuels using algae. We express FAEES (fatty acid ethyl ester synthase) in algae, and use the recombinant FAEES (fatty acid ethyl ester synthase) in the algae to synthesize biodiesel, fatty acid ethyl ester or fatty acid methyl ester, in vivo from fatty acids in the presence of ethanol/or methanol.

### FIELD OF THE INVENTION

The present invention relates to expression and application of recombinant FAEES in algae as a novel method to produce biodiesel (fatty acid ethyl esters or fatty acid methyl esters) in vivo, in the presence of ethanol or methanol.

### BACKGROUND OF THE INVENTION

FAEES is an enzyme, which can synthesize fatty acid ethyl esters/fatty acid methyl esters from fatty acids and ethanol/methanol in vivo. For example, fatty acid ethyl esters (FAEE), esterification products of fatty acid and ethanol, have been shown to be produced in HepG2 cells that release FAEE into the culture medium upon incubation with ethanol. The synthesis of FAEE was observed within 5 min of the addition of ethanol, with a plateau for FAEE synthesis after 2 h of incubation. It was also observed that FAEE are synthesized by both a microsomal FAEE synthase (FAEES), which preferentially uses fatty acyl-CoA as a substrate, and a cytosolic FAEE synthase, which accepts both unesterified fatty acid and fatty acyl-CoA as substrates with a slight preference for fatty acyl-CoA. FAEES can also synthesize FAME from methanol and fatty acids.

A continuous effort has been invested to find alternative resources for replacement of petroleum products for energy. Ethanol has been produced from corn and other materials, eg. switchgrass, and oils/fatty acids have been produced from algae. However, current efforts to produce biodiesel have been utilizing fatty acids and ethanol in vitro under weak acidic or weak basic conditions. The esterification reaction is carried out in a reaction vessel, but the efficiency of the current esterification reaction is limited and costly as the reaction is labor-intensive and time-consuming. Therefore, there is an urgent need to develop novel and innovative method to produce FAEE/FAME in a cost-effective manner.

A novel method has been developed. FAEES can be produced and utilized in algae to produce FAEE in vivo in the presence of ethanol. FAEE is released into the culture medium. Similarly, FAEES can be utilized in algae to produce FAME in the presence of methanol. FAME is released into the culture medium.

### SUMMARY OF THE INVENTION

This discovery is related to the production of bioenergy fuel in vivo, specifically related the production of biodiesel in algae. Recombinant FAEES (fatty acid ethyl ester synthase) is produced in algae, and used to synthesize biodiesel in vivo from fatty acids in the presence of ethanol/or methanol.

A novel in vivo method to produce biodiesel has been developed. FAEES can be utilized in algae to produce FAEE in the presence of ethanol. FAEE can be released into the culture medium. Similarly, FAEES can be utilized in algae to produce FAME in the presence of methanol. FAME can be released into the culture medium.

### DETAILED DESCRIPTION OF THE INVENTION

The following examples are presented by way of illustration, not of limitation.

### Example 1

Clone the cDNA of FAEES from HepG2 cells by polymerase chain reaction (PCR). We inserted the PCR-amplified FAEES cDNA to pKYLX71 using two available restriction sites, Xbal1 and Xhol 1. The obtained vector construct was transformed to Chlorella vulgaris algae cells by using standard electroporation. Five and ten days after inoculation of Chlorella vulgaris and under the selection of kanamycin, transgenic Chlorella vulgaris started to grow and positive clones were selected. The presence of FAEES cDNA in algae was confirmed with PCR.

### Example 2

We produced recombinant FAEES (fatty acid ethyl ester synthase) in algae, and used the recombinant FAEES (fatty acid ethyl ester synthase) in algae to synthesize biodiesel in vivo from fatty acids in the presence of ethanol/or methanol.

### Example 3

The recombinant Chlorella vulgaris algae grew in the presence of ethanol 1% for 10 hrs. The culture medium was harvested and analyzed by GC/MS analysis for FAEE formation.

### Example 4

The recombinant Chlorella vulgaris algae grew in the presence of 1% methanol for 10 hrs. The culture medium was harvested and FAME formation was confirmed by GC/MS analysis

### BEST MODE FOR CARRYING OUT THE INVENTION

The preferred embodiments of this invention are described herein, which includes the best mode that is known to the inventors for implementing the invention to produce biofuel in vivo. Even though the invention has been described with reference to particular embodiments, it is to be understood that these embodiments are merely illustrative of the applications and principles of the invention. Therefore, it is to be understood that many modifications may be made to the illustrative embodiments without departing from the spirit and the scope of the invention as defined in the attached claims.

### INDUSTRIAL APPLICABILITY

This invention is capable of producing biodiesel in vivo in algae and microalgae in large scale, and can be used in a variety of applications where FAEES is used in algae. These FAEE or FAME could also be used as consumer goods, pharmaceuticals, functional foods, drugs, and/or in industrial products, like biofuel.

1. A novel method to produce biodiesel, FAEE (fatty-acid-ethyl-ester) and FAME (fatty-acid-methyl-ester) in vivo.

2. The present invention relates to the ability to insert an exogenic cDNA of FAEES (fatty acid ethyl ester synthase or fatty-acyl-ethyl-ester synthase) into the algae genome and express recombinant FAEES in algae.

3. The present invention relates to utilization of recombinant FAEES (fatty acid ethyl ester synthase) in algae. Insertion of the exogenic FAEES into algae genome enables the algae to produce recombinant FAEES; but not limited to algae, it can be applied to other plants as well.

4. The application of the recombinant FAEES in algae is due to its ability to synthesize fatty acid ethyl ester in vivo from the fatty acids, using intrinsic ethanol produced by the organism.

5. The application of the this technology is due to the ability of FAEES to synthesize fatty acid ethyl ester from the fatty acids, using extrinsic ethanol, which is obtained externally.

6. The application of the technology is due to the ability of FAEES to synthesize fatty acid methyl ester from the fatty acids, using extrinsic methanol, which is obtained externally.
